(19) United States
(12) Patent Application Publication
(10) Pub. No.: US 2010/0055709 A1
(43) Pub. Date: Mar. 4, 2010

(54) Tubulin Mutation Diagnostic

(76) Inventors: Kenneth Wayne Culver, Mendham, NJ (US); Markus Wartmann, Riehen (CH); Stan Lilleberg, Omaha, NE (US); Jian Zhu, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/610,400

(22) Filed: Nov. 2, 2009

Related U.S. Application Data
(63) Continuation of application No. 11/718,957, filed on May 9, 2007, now abandoned.

(30) Foreign Application Priority Data
Nov. 12, 2004 (GB) 0425038.7

(51) Int. Cl. C12Q 1/68; C07H 21/04; C07K 14/435; C12Q 1/02

(52) U.S. Cl. 435/6; 536/23.5; 536/23.1; 530/350; 435/29

(57) ABSTRACT

The present invention relates to methods for determining the potential of a subject to respond to a particular therapeutic agent, by determining the presence of one or more nucleotide or amino acid variants in the β-tubulin gene or protein. Also provided are methods for treating subjects whose potential to respond to a therapeutic agent has been evaluated. For use in the methods of the invention, there are provided variants of the β-tubulin gene. variants of the β-tubulin protein, nucleic acid molecules and agents which bind to the variant β-tubulin nucleic acid molecules and variant β-tubulin protein, respectively, and kits comprising the same.

### FIELD OF THE INVENTION

The present invention relates to methods for determining the potential of a subject to respond to a particular therapeutic agent, by determining the presence of one or more nucleotide or amino acid variants in the β-tubulin gene or protein. Also provided are methods for treating subjects whose potential to respond to a therapeutic agent has been evaluated. For use in the methods of the invention, there are provided variants of the β-tubulin gene, variants of the β-tubulin protein, nucleic acid molecules and agents which bind to the variant β-tubulin nucleic acid molecules and variant β-tubulin protein, respectively, and kits comprising the same.

### BACKGROUND OF THE INVENTION

β-tubulin dimerises with the related protein, α-tubulin, to form heterodimers which polymerise into microtubules. To fulfil their roles in cell motility, intracellular transport and maintenance of cellular architecture, the microtubules are dynamic structures, being able to shrink or grow as required by the cell. This dynamicity is achieved by ongoing polymerisation and depolymerisation of the α- and β-tubulin heterodimers, with heterodimers being incorporated into a microtubule when it requires lengthening, and heterodimers being released into the cytoplasm when the microtubule requires shortening.

One of the most important roles of the microtubules is in cell division, where they form the mitotic spindle which mediates movement of the chromosomes during mitosis.

Numerous proteins are associated with the microtubules, and are termed Microtubule Associated Proteins, or MAPs. These proteins bind to the microtubules, and influence both their stability and motility within the cell. For example, MAP2 and MAP4 stabilise the polymerised microtubule, whereas others, including stanthim, induce depolymerisation.

α- and β-tubulin are related proteins, encoded by a multigene family. To date, six isotypes of β-tubulin have been identified, which are highly conserved. The human isotype M40 is expressed ubiquitously in all cells including cancer cells, and is the most prevalent isoform. In addition to the various isotypes, pseudogenes of β-tubulin have also been found.

As a result of its crucial role in forming the mitotic spindle for mitosis, tubulin has long been recognised as an important target for drugs which disrupt mitosis, and therefore cell division. These drugs, often referred to as anti-mitotic agents, are important in treatment of conditions in which cell division requires inhibiting, for example, cancer.

Four main families of anti-mitotic agents have been identified, and proposed for use in the treatment of cancers. These are the taxanes, which are derived from the yew tree, and include paclitaxel (Taxol™) and docetaxel; the vinca alkaloids, derived from the periwinkle plant; the epothilones from soil bacteria; and the dolastatins. The taxanes and epothilones both stabilise the microtubule, preventing depolymerisation. The vinca alkaloids and dolastatins, on the other hand, encourage depolymerisation, thus reducing the stability of the microtubules. Evidence suggests that these agents mediate their effects by binding to tubulin, and so maintenance of functional binding sites on tubulin would seem to be crucial to their anti-mitotic effect (Nishio et al, Anti-Cancer Drug Design (1999) 14 133-141).

Despite their potential for treatment of conditions such as cancer, increasing resistance to anti-mitotic agents is being observed. This is thought to be caused by various factors, including insufficient accumulation of the agent in the cell, altered binding to tubulin, increased metabolism of the agent, and altered MAPs. To investigate the nature of this resistance to anti-mitotic agents, resistant cell lines have been established and used as research tools. In some such cell lines, changes to tubulin, including mutations to the β-tubulin gene and protein have been observed. Upon review of the mutations identified to date, Berrieman et al (The Lancet, Vol 5 March 2004) concluded that only those genetic variations which change the protein sequence could be expected to have any effect on responsiveness to anti-mitotic drugs. Such genetic mutations may be inherent to an individual, or could conceivably arise at the site of the cancer during treatment.

Nucleotide polymorphisms occur, on average, once every kilobase across the genome and account for much of the genetic diversity in tumour populations. Whilst for some time it has been appreciated that such genetic diversity may affect gene expression and therefore account for susceptibility or onset of disease, it is now recognised that this genetic diversity may also play a significant role in determining an individuals responsiveness to a particular treatment.

Polymorphisms within the genome may be manifested as restriction fragment length polymorphisms, tandem repeats, hypervariable regions, mini-satellites, di- or multi-nucleotide repeats, insertion elements and single nucleotide polymorphisms. The latter are referred to as SNPs, and are single positions at which genetic variation occurs, by way of nucleotide insertion, deletion or substitution. The different variations at a SNP site are referred to as alleles, with the first identified allele being the reference allele or wild type allele. Depending upon the number of nucleotide alternatives at the SNP site, it may be di- or tri-allelic. For example, where the wild type allele is a “T” residue, the other alleles may contain a “C”,“G” or “A” at that site. Single nucleotide polymorphisms may result in corresponding changes to the amino acid sequence. For example, substitution of a nucleotide residue may change the codon, resulting in an amino acid change. Similarly, the deletion or insertion of three consecutive bases in the nucleic acid sequence may result in the insertion or deletion of an amino acid residue.

A SNP which occurs within the protein coding sequence of a gene may thus affect the protein structure or function. The effect may be neutral, beneficial or detrimental, depending upon the circumstances. SNPs occurring in the non-coding 5′ or 3′ untranslated regions of a gene may not affect protein sequence, but may exert phenotypic effects by affecting RNA transcription, processing and/or translation. A polymorphism may affect more than one phenotypic trait or may be related to a specific phenotype.

Diseases such as cancer can be highly invasive, and often need to be treated quickly and effectively in order to maximise the chances of survival. Many anti-cancer therapies are, by their very nature, harsh with unpleasant side effects. In addition, they are costly, and place a financial burden on the hospital. For these reasons, it is increasingly desirable to be able to identify those therapeutic agents which a subject is most likely to respond to, preferably before treatment begins.

Understanding molecular variations in drug targets, for example by sequencing genes and characterising polymorphisms, will give a better understanding of how variations in the effectiveness of drugs between subjects arise. Ultimately, understanding the basis for such variation will allow better matching of drugs subjects, and thus improved treatment and increased cost-effectiveness of health care.

The present invention aims to make use of newly identified polymorphisms in the β-tubulin gene and protein, and provide a test for identifying those subjects who are likely to be resistant or hypersensitive to a therapeutic agent. In this way, the present invention aims to overcome the shortcomings in the art, and enable treatment programs to be tailored to individual needs.

### DESCRIPTION OF THE INVENTION

Thus, in a first aspect of the invention, there is provided a method of determining the potential of a subject to respond to a therapeutic agent, the method comprising determining the presence of an amino acid variant at one or more of positions 263, 268, 270, 274, 276, 293, and 364 of the β-tubulin protein as represented in FIG. 1. The presence or absence of a variant is indicative of resistance or hypersensitivity to the therapeutic agent.

The presence of an amino acid variant may be determined by examination of the amino acid sequence of the protein or the nucleic acid sequence which codes for the protein. Thus, in a second aspect of the invention, there is provided a method of determining the potential of a subject to respond to a therapeutic agent, the method comprising determining the presence of a nucleotide variant, in a nucleic acid sequence which codes for β-tubulin, which causes an amino acid variation at position 263, 268, 270, 274, 276, 293 and/or 364 of the β-tubulin protein as represented by FIG. 1. Preferably, the method comprises determining the presence of a nucleotide variant at one or more of positions 787, 803, 808, 821, 827, 878 and/or 1091 of the nucleic acid sequence encoding β-tubulin as represented by FIG. 2.

In a third aspect, the present invention provides a method of treating disease in a subject, the method comprising (a) determining the potential of a subject to respond to a therapeutic agent, by determining the presence of an amino acid variant at one or more of positions 263, 268, 270, 274, 276, 293, and 364 of the β-tubulin protein as represented in FIG. 1, or the presence of a nucleotide variant, in a nucleic acid sequence coding for β-tubulin, which causes an amino acid variation at one or more of the above positions, wherein the presence or absence of a nucleotide or amino acid variant is indicative of resistance or hypersensitivity to the therapeutic agent; and (b) administering a therapeutic agent to which the subject is not resistant.

In a fourth aspect of the invention, there is provided an isolated or recombinant nucleic acid molecule comprising a nucleic acid sequence encoding β-tubulin, or a nucleic acid sequence complementary thereto, having a nucleotide variant either which causes a variant at position 263, 268, 276 and/or 293 of the β-tubulin protein as represented by FIG. 1 or is at a position corresponding to 787, 803, 827 and/or 878, of the nucleic acid sequence represented by FIG. 2; and fragments thereof.

In a fifth aspect, nucleic acid sequences are provided which are able to hybridise to a nucleic acid sequence encoding β-tubulin as represented in FIG. 2, or to a strand complementary thereto. These nucleic acid sequences are preferably able to hybridise to a nucleotide variant which causes a variation at positions 263, 268, 276 and/or 293 of the β-tubulin protein represented by FIG. 1, or to a region flanking such a variant, to enable amplification of the variant site.

In a sixth aspect, there is provided an isolated β-tubulin protein as represented by FIG. 1, having an amino acid substitution at position 263, 268, 276 and/or 293.

In a seventh aspect, there are provided protein binding agents specific for a variant at positions 263, 268, 276 and/or 293 of the β-tubulin protein represented by FIG. 1.

In an eighth aspect of the invention, there is provided a method of screening for suitable therapeutic agents, comprising

a) providing cells comprising an amino acid variant at one or more of positions 263, 268, 270, 274, 276, 293 and 364 of the β-tubulin protein as represented by FIG. 1; or nucleic acid sequences encoding β-tubulin and having a variant which cause one or more of the amino acid variants; and

b) exposing the cells to a therapeutic agent; and

c) monitoring the polymerisation and/or depolymerisation of β-tubulin in the cells upon exposure to the therapeutic agent;

wherein suitable therapeutic agents are those which are capable of affecting the polymerisation and/or depolymerisation of β-tubulin in the cells.

Finally, the present invention provides a kit for use in a method of the invention comprising nucleic acid sequences as herein described, and/or protein binding agents, for detecting the nucleotide or amino acid variants of β-tubulin as described herein, together with a reference chart detailing the correlation between nucleotide or amino acid variants and potential of the subject to respond to particular therapeutic agents.

### DESCRIPTION OF THE FIGURES

The present invention will be described in detail below, with reference to the Figures, in which:

FIG. 1 shows an in-frame translation of the cDNA sequence of the Human M40 β-tubulin cDNA. The protein extends from the first start methionine residue to the first stop residue.

The variant amino acids are shown in bold type, and all possible start methionine residues and stop sites are shown in italic type.

FIG. 2 shows the cDNA transcript of the β-tubulin gene, where suitable primers bind to the italic sequences, and the variant nucleotide sites are shown in bold type.

FIGS. 3 and 3a show the amplicons of the HM40 β-tubulin gene, with the sequences to which the primers bind shown in italic type, the exons in underlined type and the introns in normal type.

FIG. 4 shows the positions of the variants with respect to fragments of AC006165.

### DETAILED DESCRIPTION OF THE INVENTION

The present invention is based upon the identification of polymorphisms in the Human M40 β-tubulin gene, which are believed to affect ability to respond to therapeutic agents, such as those which mediate their action via tubulin. The genomic sequence of the HM40 Human β-tubulin gene has been isolated, and is provided in Genbank Accession Number AC006165. This 44,118 base pair sequence of chromosome 6 comprises four exons which encode a protein of 444 amino acids and approximately 49 kDa in weight. In a cDNA or mRNA transcript of β-tubulin, the four exons span nucleotides 1 to 57, 58 to 166, 167 to 277, and 278 to 1335.

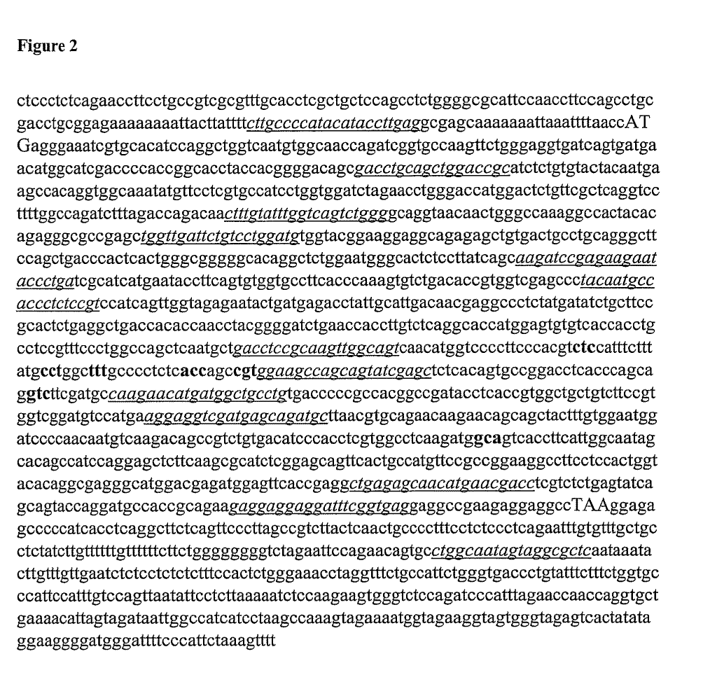

The β-tubulin protein is that encoded by the cDNA sequence of FIG. 2, and which corresponds to the sequence of Uniprot Accession Number 05218.

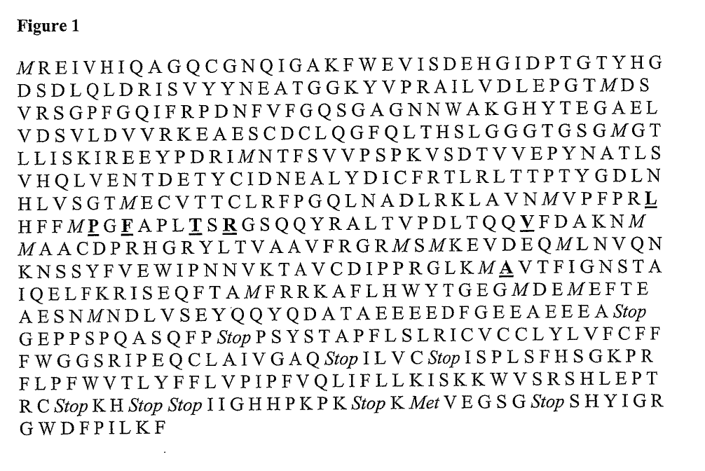

The variant nucleotides and amino acids of the present invention have each been assigned a positional reference with respect to FIGS. 1 and/or 2. The nucleotides variants are located at positions 787, 803, 808, 820, 821, 827, 878 and 1091 of the cDNA sequence shown in FIG. 2. These polymorphisms result in the nucleotide changes 787C>A, 803T>A, 808T>G, 821C>T, 820A>C, 827G>A, 878T>A and 1091G>A respectively, and are non-synonymous, resulting in the corresponding amino acid changes 263 L>I, 268P>H, 270F>V, 274T>I 274T>P, 276R>H, 293V>D, and 364A>T in the β-tubulin protein of FIG. 1.

The β-tubulin gene is transcribed in the reverse orientation to the sequence provided in AC006165. For ease of reference, therefore, the nucleotide variants of the present invention have been described with respect to the cDNA sequence. The corresponding positions of the nucleotide variants in the genomic sequence can be readily ascertained using sequence comparison methods, for example BLAST analysis. This latter method has revealed the nucleotide variants at positions 787, 803, 808, 820, 821, 827, 878 and 1091 of the cDNA sequence to correspond to positions 35273, 35257, 35252, 35239, 35233, 35182 and 34970 of the reverse, inverted sequence of AC006165.

Resistance or hypersensitivity to anti-mitotic drugs is thought to arise due to alteration in the binding of the agents to β-tubulin. This is often the case where sequence variation causes changes to the binding sites of the drugs. The amino acid variants at positions 270 and 274 of FIG. 1 lie within the binding site for epothilones and/or taxanes on β-tubulin. These mutations are believed to confer resistance to these classes of anti-mitotic agent. (Wartmann et al; Curr. Med. Chem—Anti Cancer Agents (2002) 2 123-148).

The potential of a subject to respond to a particular therapeutic agent is a measure of the subject's resistance or hypersensitivity to that therapeutic agent. Thus, by screening a subject for one or more variants of the β-tubulin gene or protein as described herein, it is possible to predict whether they will be resistant or hypersensitive to a particular therapeutic agent—ie, the subject's potential to respond can be assessed. The present inventors have found that variants of the β-tubulin gene which confer resistance to a β-tubulin stabilising agent also confer hypersensitivity to β-tubulin destabilising agents, and visa versa. Thus, by analysing the variants described herein, it is possible to determine a subject's ability to respond to treatment both in terms of resistance and hypersensitivity to different classes of agent.

The potential to respond to a therapeutic agent is now believed, in part, to be an inherent characteristic, generally dependant upon the genetic profile of a subject, in particular those cells which are in a diseased state, for example tumour cells where the disease is cancer. By determining the genetic profile of a subject, it is possible to obtain an indication of whether they are inherently capable of responding to the therapeutic agent. Thus, if based upon the genetic profile a subject is considered to be capable of responding to a therapeutic agent, and yet no clinical response is achieved, it enables other factors affecting responsiveness to be analysed. This will effectively reduce the amount of trial and error involved in treating many diseases, such as cancer.

The expected clinical response of a subject to a therapeutic agent may be measured in terms of improvement in condition, reduction in adverse symptoms, and/or a slow down in progression of disease. For example where the disease is cancer, the clinical response will include reduction in size of tumour, malignancy or other non-tumour related symptoms.

The degrees of resistance and/or hypersensitivity to any particular therapeutic agent may differ depending upon the genetic profile. Thus, analysis of the variants of the β-tubulin gene or protein as described herein can be used to indicate not only whether a subject has the ability to respond to a particular therapeutic agent, but also the degree of response which can be expected.

In terms of resistance, where this is complete a subject is deemed unlikely to be able to respond to the therapeutic agent in any capacity for the particular disease being treated. Partial resistance, however, in this context means that the subject may respond to the therapeutic agent, although to a lesser degree than if he were not resistant at all. Similarly, hypersensitivity in this context is any increase in positive clinical response to a therapeutic agent, compared to that which would be expected if the subject was not genetically hypersensitive.

The level of resistance or hypersensitivity of a subject, as determined by his genetic profile, will assist in determining which agent will be effective, and at what dose it should be administered. It may be desirable to compare the subject's genetic profile to a “standard” in order to assess the degree of resistance or hypersensitivity, and thus the dose of therapeutic agent. Such standards may be created by averaging the degree of resistance or hypersensitivity for a number of subjects and particular genetic profiles.

It is generally preferable to determine the potential of a subject to respond to a particular therapeutic agent prior to the commencement of treatment, such that an appropriate therapeutic agent may be selected, depending upon the genetic profile of the subject. Thus, the method may be conducted at any suitable time, preferably after diagnosis of disease, and preferably prior to the commencement of treatment.

However, as it has been observed that the responsiveness of a subject can change during treatment, it may be desirable to monitor this during the treatment program, so that the treatment can be changed accordingly, if necessary. This ensures that, as far as possible, only treatments to which the subject is able to respond are administered. Monitoring in this way can be conducted at any time, although preferably when a subject begins to show signs of resistance, or at specified time intervals during the treatment program, for example daily, weekly or monthly.

The present invention is preferably carried out on a suitable sample, removed from the subject. A suitable sample is one which contains nucleic acid or protein. Preferably it is easily removed from a patient, without the need for surgery, or is archival tissue which has been previously removed from a patient, for example during a biopsy. Preferred samples include whole blood, semen, urine, faeces, saliva, skin, hair, tears, or buccal cells. Where it is desired to analyse cDNA, mRNA or protein, the sample is preferably one in which the β-tubulin gene is expressed, for example, or which contains β-tubulin protein, or gene products. From the sample, nucleic acid or protein may then be extracted, using methods available to persons skilled in the art.

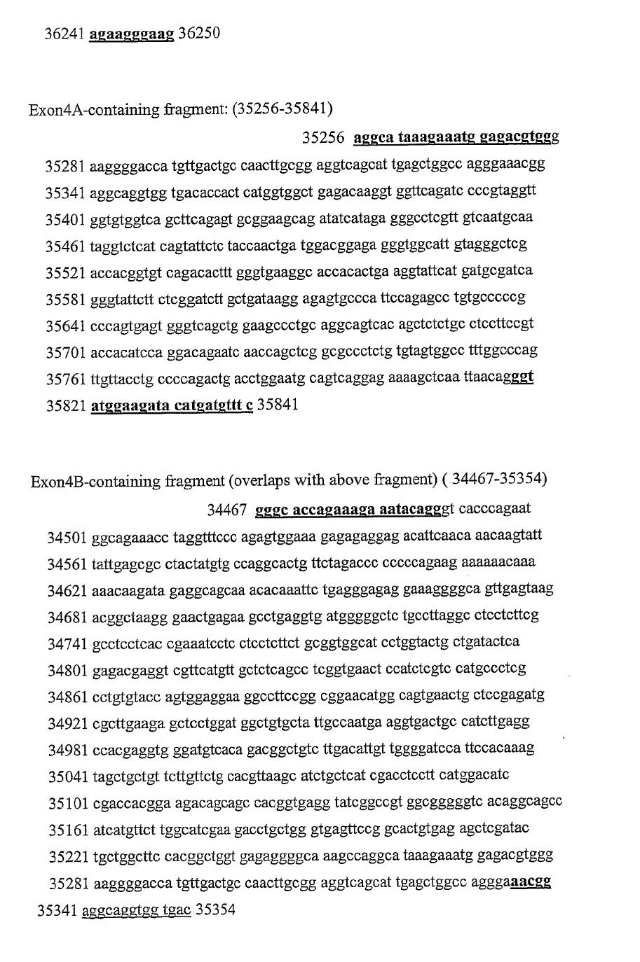

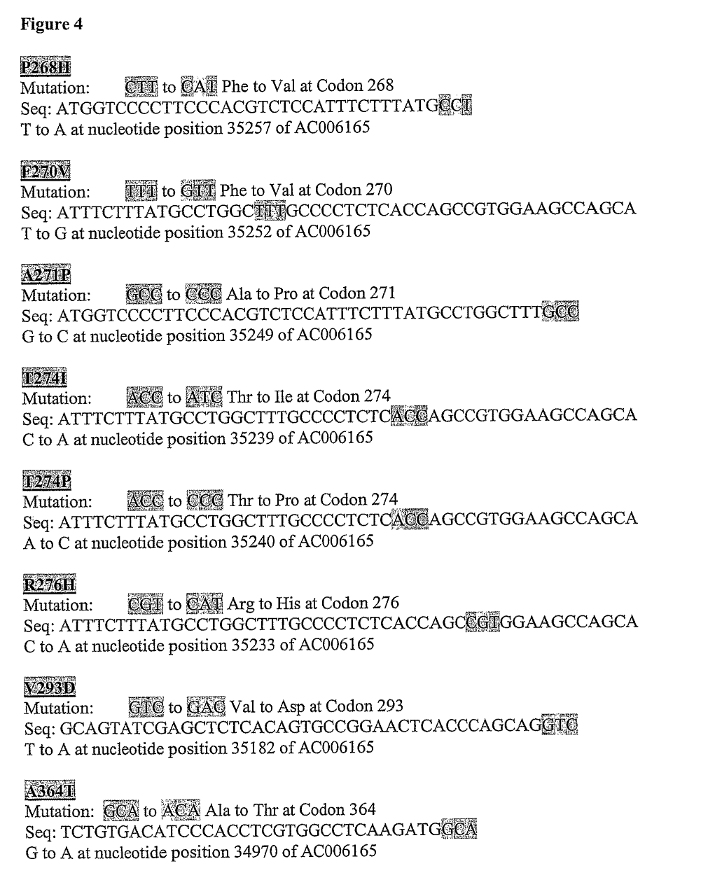

In the present invention, it is apparent that it is the position of the above-mentioned variants in the β-tubulin cDNA and protein, and the uses thereof, which are the novel and limiting feature. The reference to the sequences of FIGS. 1 and 2 are for guidance only, in order to confirm that the variants are in β-tubulin. It is envisaged that the nucleic acid or protein sequence to be examined in the present invention need not be identical to that of FIG. 1 or 2. Thus, reference herein to the β-tubulin gene includes genomic, cDNA or RNA sequences, which may differ in sequence to that of FIG. 2, for example isotypes of β-tubulin, such as those described in Berrieman et al (supra). Reference to β-tubulin protein includes protein sequences which may differ in that of FIG. 1. Where the sequence to be examined is not identical to that of FIG. 1 or 2, the expected positions of the variants can be readily determined by aligning the sequence with that of FIG. 1 or 2, or other available clones, using methods available in the art, for example using computer programs such as DNASIS, Word Search or FASTA. Alignment of the nucleotide variants with the genomic sequence of AC006165 may be done using BLAST analysis with the sequences of FIG. 4.

In the present invention, a “variant” is a nucleotide or amino acid residue which differs from the residue of the reference sequences disclosed in FIGS. 1 and 2 respectively, at any particular position. The variants may be nucleotide or amino acid deletions, substitutions or insertions, and may also be referred to as polymorphisms or mutations. For nucleotide variants which occur on the complementary DNA strand of the sequence of FIG. 2 or AC006165, a variant at a particular position is a residue which is not complementary to the wild type residue with which it is paired. If the residue at a particular position on the DNA strand shown in FIG. 2 is a variant, then the residue with which it is paired at the opposite strand may or may not be complementary, depending on whether it is the wild type residue or a variant. For ease of reference, where the variants are insertions or deletions in the genetic sequence, the numbering of FIG. 2 has been maintained.

By “determining the presence of a variant” of the β-tubulin gene or protein is meant ascertaining the sequence of the gene or protein at a particular position, in order to determine which specific amino acid or nucleotide residue is present at that site. Any suitable method for determining the presence of one or more amino acid or nucleotide variants of β-tubulin may be used.

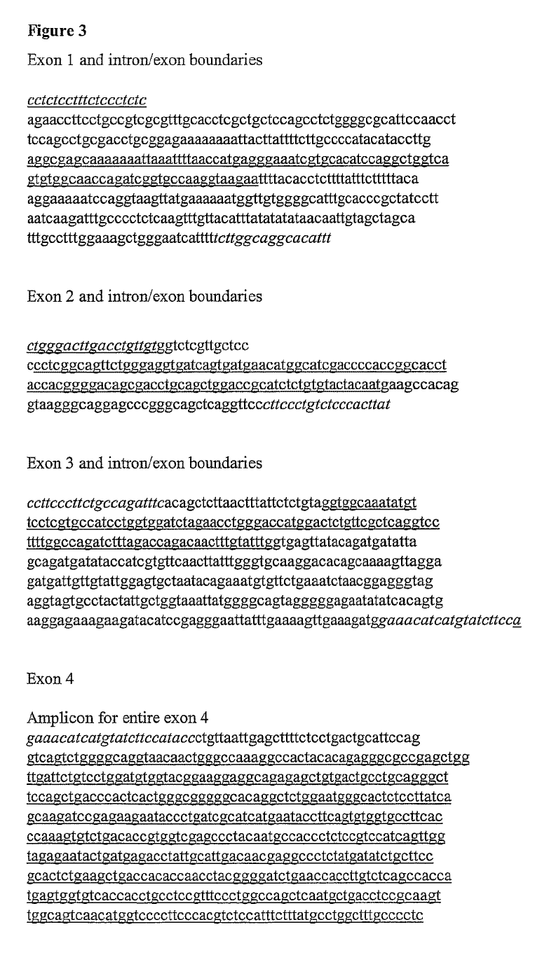

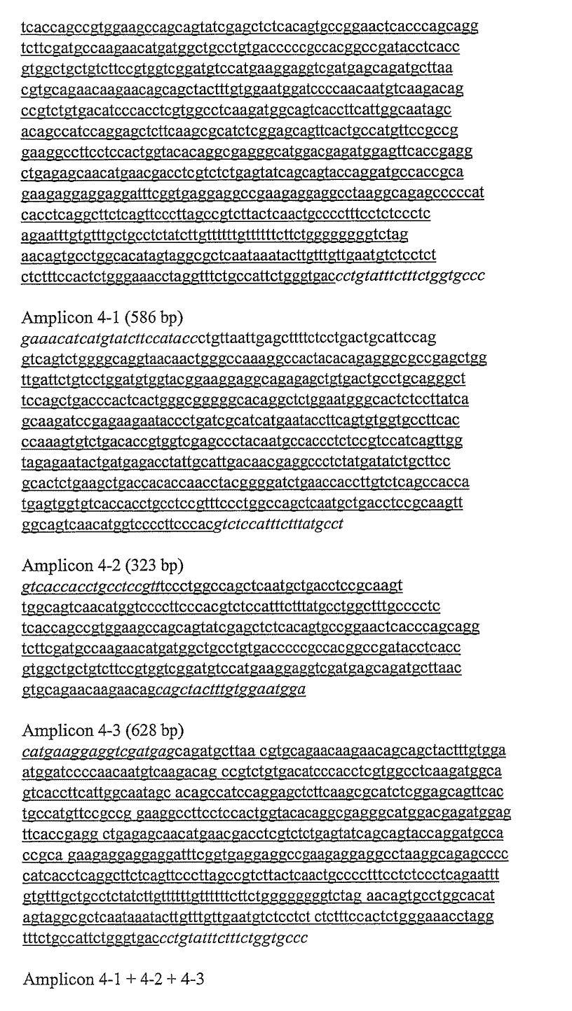

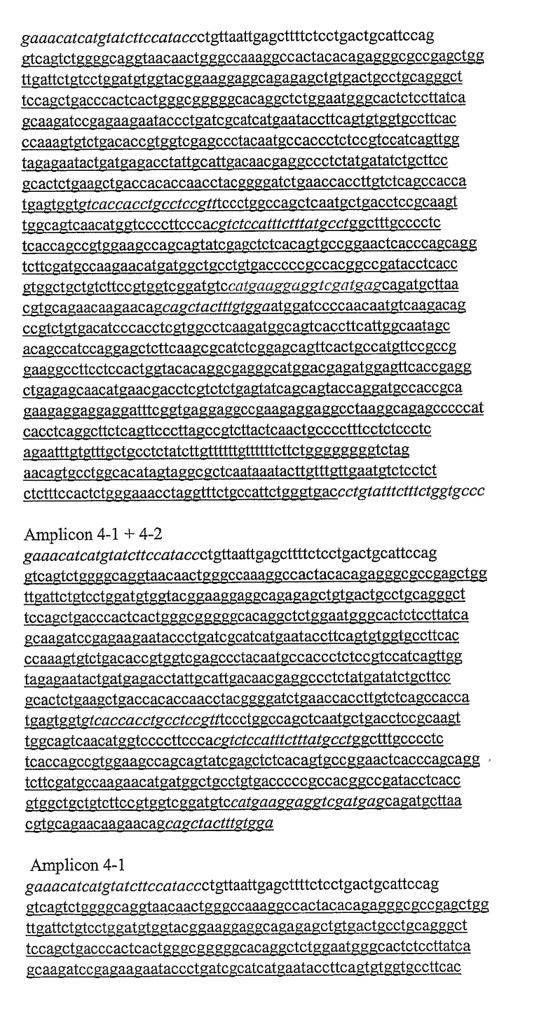

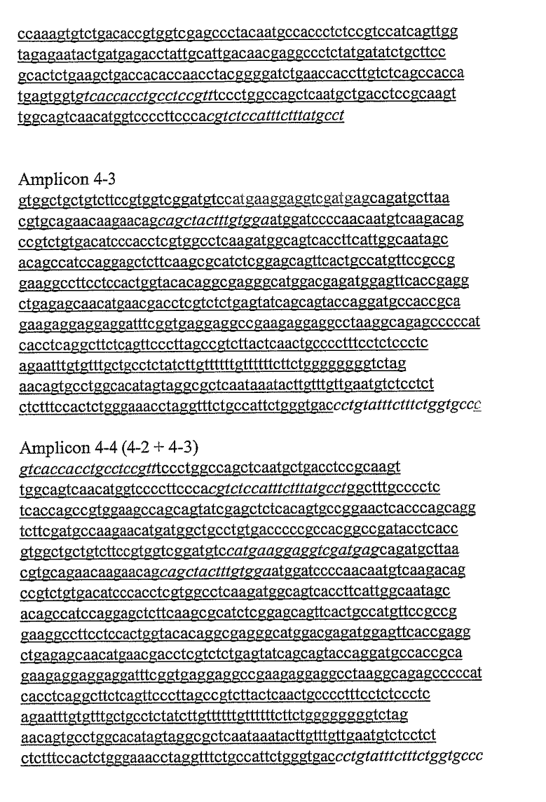

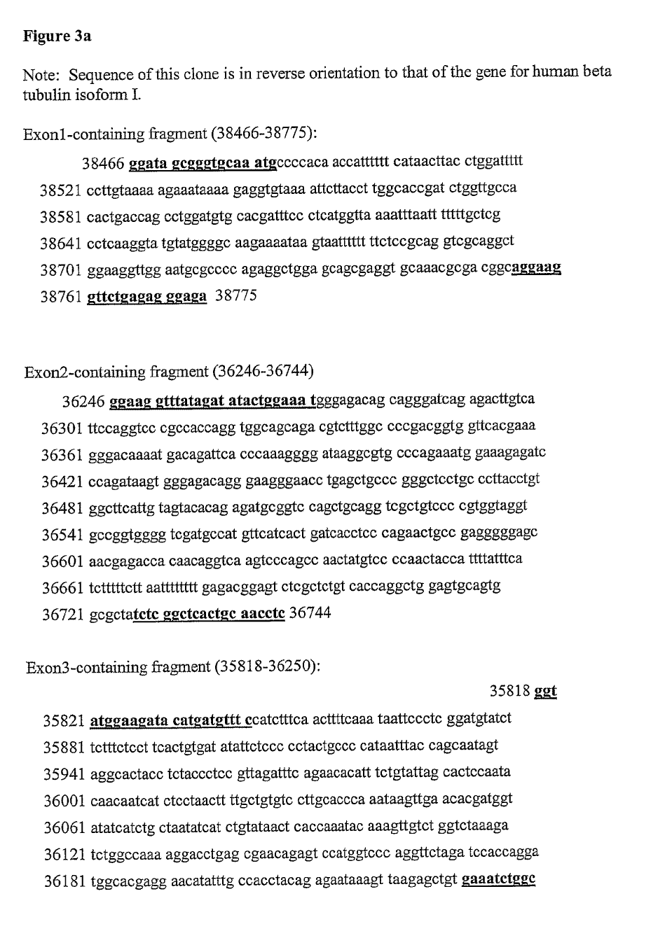

The methods of the invention do not have to be carried out on the full length β-tubulin gene, gene product or protein, but may for convenience be carried out on a fragment thereof which comprises one or more of the nucleotide or amino acid variants. The fragments may be of any size, so long as it is possible to ascertain that the fragment is a β-tubulin fragment, and/or determine the presence of one or more variants therein. In order to determine whether a fragment is of β-tubulin, or which part of the gene it is derived from, the fragments can be aligned with the sequences of FIG. 1 or 2 or the genomic sequence of AC006165, and compared for example with computer programs such as DNASIS (Hitachi Engineering Inc), Word Search or FASTA (Genetic Computer Group, Madison Wis.). In this way, the positional references used for variants of the sequences of FIGS. 1 and 2 can be maintained when referring to fragments of these sequences. Preferred fragments contain at least one exon of the gene, and are shown in FIG. 3. Suitable fragments will preferably be between 10 and 400 nucleotides in length, preferably at least 10, 50, 80, 100, 200, 300 or 350 nucleotides in length. Preferably, the fragments will comprise or encode at least one variant of β-tubulin described herein.

When determining nucleotide variants, it is generally preferred to first amplify the nucleic acid sample, and this may be done using any available technique, such as PCR or PCR based technologies, ligase mediated reaction, transcription amplification, self sustained sequence replication and nucleic acid based sequence amplification. The latter two methods involve isothermal reactions based upon isothermal transcription. Preferably, due to the size of the β-tubulin gene, fragments of the gene are amplified and screened for variants. Suitable fragments of the gene to be amplified are selected on the basis of criteria including % helicity, and analysis conditions such as salt concentration, length of amplicons, % of GC residues and mobile phase concentration. Any suitable portion of the gene may be amplified, preferably the amplicons comprising one or more variant sequences. Preferably, the amplicons may comprise one or more exon sequences, or parts thereof. Examples of amplicons sequences are shown in FIGS. 3 and 3a. The preparation of suitable primers for amplifications of portions of the β-tubulin gene will be within the capabilities of a person skilled in the art. When amplifying the β-tubulin gene, it is preferable to do so in a manner which will reduce the concurrent amplification of pseudogenes. Suitable methods will be known to persons skilled in the art. A preferred method involves placing one amplification primer within an intron of the β-tubulin gene, preferably the intron between the third and fourth exons. Any suitable intron based primer may be used, and can be designed by a person skilled in the art using the primer design criteria disclosed herein, or methodology available in the art. Preferred intron based primers for the prevention of amplification of the β-tubulin pseudogenes included are those described in the examples.

Suitable methods for determining the presence of one or more of the nucleotide variants of the invention include, but are not limited to, sequencing of PCR products, direct probing direct cloning and sequencing, Allele Specific Hybridisation, Mutant Allele Specific Amplification (MASA), RFLP, polymerase mediated primer extension, single base extension, and rolling circle amplification following allele specific ligation.

The preferred method for determining the presence of a variation is MASA. Thus in a preferred embodiment of the present invention, the method of predicting the ability of a subject to respond to a particular therapeutic agent comprises amplifying the β-tubulin gene with a pair of primers, one being specific for the variant allele and/or amplifying the β-tubulin gene with a second pair of primers, one being specific for the wild type of reference allele at the same position. If amplification occurs using the primer specific for the variant allele, then the variant is present. Conversely, if amplification occurs using the primer specific for the wild type allele, then the wild type allele at that position is present. Amplification in both reactions indicates a heterozygous genotype at that position.

Preferred primers for use in MASA according to the invention may be designed using computer programs and methodology available in the art. Preferred primers are detailed in the examples.

In a preferred embodiment, amplification of the wild type allele may be prevented, or blocked, by the use of a third primer which is specific for the wild type allele, but which is unable to mediate amplification. This primer preferably is similar in sequence to the mutant allele specific primer, with the exception that it is instead specific for the wild type allele, and additionally is unable to mediate primer extension. For example, this third primer may be blocked at its 3′ end, and thus is unable to mediate primer extension, and amplification. Suitable primers will be readily designed by persons skilled in the art taking into account the gene sequence and wild type allele.

When determining amino acid variants, this is preferably done by detecting binding of protein binding agents to the variant amino acid. Suitable binding agents include aptamers, and antibodies. Preferably, to assist detection of binding to the variant amino acid, the binding agent will be labelled, or coupled to a secondary antibody, enzyme or molecule, which can be detected, as described herein.

In a preferred embodiment of the present invention, the β-tubulin gene or protein may be screened to establish if any variants are present, before such variants are determined. This screening step will serve to accelerate the agent selection and treatment process, especially for those subjects who are found not to have any variant sequences, and therefore can begin treatment immediately without requiring further tests to determine which variants are present. Suitable screening methods include those which rely upon heteroduplex analysis. Preferred methods include mismatch detection, for example using proteins such as E. coli mutS or riboprobes, gel electrophoresis, denaturing high performance liquid chromatography (DHPLC) and single stranded conformation polymorphism analysis (SSCP). In a most preferred embodiment of the invention, DHPLC is used to determine the presence of any variation in the β-tubulin gene or protein. Thus, a method of the invention will preferably comprise the additional steps of:

(a) separating the strands of the nucleic acid sample to be tested;

(b) forming a duplex with a strand known not to contains any variant nucleotides at one or more of positions 787, 803, 808, 820, 821, 878 and/or 1091 of FIG. 2;

(c) producing a melting profile for the duplex, wherein profile indicates the presence of one or more variants in the strand being tested.

Preferably, the DHPLC is performed using the WAVE® System (Transgenomic) or the Helix System (Varian).

It may be possible to determine the presence of one or more of the amino acid or nucleotide variants of the invention indirectly, by determining the presence of another variant which is in linkage disequilibrium with one or more of the afore-mentioned β-tubulin nucleotide variants. Variant sites are in linkage disequilibrium when the presence of one variant increases the chances of the second variant being present. Therefore, by determining the presence of one variant, it is possible to predict that the second variant will be present, and to proceed on that basis without actually confirming the presence of the second variant. It is apparent that variants which may be in linkage disequilibrium with one or more of the nucleotide variants of the invention may be known, and may be located in other genes or parts of the genome otherwise unrelated to β-tubulin.

The present invention will be suitable for assessing whether a subject is able to respond to a therapeutic agent which targets, or mediates its action via, β-tubulin. Such therapeutic agents include those which stabilise tubulin for example taxanes, epothilanes, discodermolide, laulimalide, peloruside and taccalomolides, and those which destabilise tubulin, for example, colchicines, and other colchicine-site binders, vinca alkaloids and others. Anti-mitotic agents include the epothilanes, vinca alkaloids and dolastatins.

Once the genetic profile of a subject has been ascertained, appropriate therapeutic agents may be selected, and doses established. Thus, where the genetic profile suggests that a subject is completely resistant to a particular agent, this may be deleted as a candidate drug. Other drugs, which the subject does not show resistance to, or is likely to be hypersensitive to, may be chosen in its place. Where the genetic profile suggests that a subject is partially resistant or hypersensitive to a drug, this may affect the dosage of the drug to be administered. For example, the dosage may be increased or reduced, or the frequency of administration may be changed.

Thus, the present invention also provides a method for treating a subject, preferably once the potential to respond to a therapeutic agent has been determined as described herein. Thus, once a genetic profile has been generated, the method may preferably comprise administering a therapeutic agent which the patient is not completely resistant to, or is hypersensitive to.

Thus, the invention provides a therapeutic agent for use in treating a disease in a subject who is not completely resistant to said therapeutic agent.

Alternatively, the invention provides use of a therapeutic agent in the manufacture of a medication for the treatment of a disease in a subject who is not completely resistant to the agent.

The present invention may be suitable in assessing potential to respond to, or treatment of, any disease which results from de-regulation of a process involving β-tubulin or microtubules, or which is treated by targeting β-tubulin. Such diseases include cancers, such as breast, ovary, lung, pancreas, kidney, liver, uterus, stomach, bowel, oesophagus, blood, bladder, bone, cervix, skin and any other organ. Preferably, the method is used in non-ovarian cancer, such as breast cancer.

The present invention also provides an isolated or recombinant nucleic acid molecule comprising a nucleic acid sequence encoding β-tubulin, or a nucleic acid sequence complementary thereto, having a nucleotide variant which causes a variant at position 263, 268, 270, 274, 276, 293 and/or 364 of β-tubulin protein as represented by FIG. 1, or at a position corresponding to 787, 803, 808, 820, 821, 827, 878 and/or 1091 of FIG. 2; and fragments thereof.

The nucleic acid may be DNA, RNA or PNA.

The nucleic acid molecule may comprise the full β-tubulin gene of AC006165 or transcript, as represented by FIG. 2, or fragments thereof. The fragments preferably comprise at least one of the variants at one of the afore-mentioned positions. Preferred fragments are 10 to 400 nucleotides in length, preferably 20, 50, 80, 100, 200, 300, or 350 nucleotides.

The nucleic acid molecule may be provided in a vector, to enable in vivo or in vitro expression. Suitable vectors will be known to those in the art, and include pBluescript II, Lambda Zap, and pCMV-Script. The nucleic acid molecule may be operably linked to one or more additional nucleic acid sequences including regulatory sequences such as a promoter and enhancers, and other sequences which facilitate cloning or expression, such as an origin of replication, one or more restriction sites, markers, ribosome binding sites, RNA splice sites, transcription termination regions, polymerisation sites and 3′ polyadenylation sites, and sequences which facilitate detection and purification of the nucleic acid molecule or product, for example tags such as GFP.

The particular choice of regulatory, and other, sequences will largely depend upon the expression system used, and may be selected from any available in the art.

Also provided are nucleic acid sequences which hybridise to the β-tubulin gene, and preferably to an allele of a variant nucleotide of the invention. Such anti-sense sequences are useful as probes or primers for detecting alleles, or in the diagnosis or treatment of disease.

To be useful as a probe in the methods of the present invention, an anti-sense nucleotide sequences must be capable of discriminating between different alleles of variant nucleiticles of the β-tubulin gene, using methods available in the art. Thus, the probe will preferably hybridise to a region including a variant site, and the probe will comprise an exact complement of the variant site at that position.

Such anti-sense sequences which are capable of specific hybridisation to detect a single base mis-match may be designed according to methods known in the art and described in Maniatis et al., Molecular Cloning: A Laboratory Manual 2^{nd} Edition (1989), Cold Spring Harbor, N.Y. and Berger et al., Methods in Enzymology 152: Guide to Molecular Cloning Techniques (1987) Academic Press Inc. San Diego, Calif.; Gibbs et al., Nuc Acids Res., 17:2437(1989); Kwok et al., Nucl Acids Res 18:999; and Miyada et al., Methods Enzymol. 154: 94 (1987). Variation in the sequence of these anti-sense sequence is acceptable for the purposes of the present invention, provided that the ability of the anti-sense sequence to distinguish between variant alleles is not compromised. For primer sequences, variation is acceptable, provided its ability to mediate amplification of the selected region is not compromised. Preferably, a primer sequence will hybridise to the β-tubulin gene under stringent conditions which are defined below.

In relation to the present invention, “stringent conditions” refers to the washing conditions used in a hybridisation protocol. In general, the washing conditions should be a combination of temperature and salt concentration so that the denaturation temperature is approximately 5 to 20° C. below the calculated Tₘ of the nucleic acid under study. The Tₘ of a nucleic acid probe of 20 bases or less is calculated under standard conditions (1M NaCl) as [4° Cx(G+C)+2° Cx(A+T)], according to Wallace rules for short oligonucleotides. For longer DNA fragments, the nearest neighbour method, which combines solid thermodynamics and experimental data may be used, according to the principles set out in Breslauer et al., PNAS 83: 3746-3750 (1986).

The optimum salt and temperature conditions for hybridisation may be readily determined in preliminary experiments in which DNA samples immobilised on filters are hybridised to the probe of interest and then washed under conditions of different stringencies. While the conditions for PCR may differ from the standard conditions, the Tₘ may be used as a guide for the expected relative stability of the primers. For short primers of approximately 14 nucleotides, low annealing temperatures of around 44° C. to 50° C. are used. The temperature may be higher depending upon the base composition of the primer sequence used.

Probes may be produced synthetically or by a process called nick-translation. In order to enable visualisation of the probe after binding to a region of the β-tubulin gene, the probe is preferably labelled, for example using radiolabels, enzymes, fluoro labels, and biotin-avidin conjugates. Once bound to the β-tubulin nucleic acid, the probe can be detected using a method which enables detection of the label. For example, where the label is a radio-label, the probe may be detected using an autoradiograph.

Preferred primers which enable amplification of part of the β-tubulin gene, for example as described above, may be readily designed by persons skilled in the art. Preferably, such primers will bind to intronic regions, closely flanking the exon sequences or within the exon sequences. Suitable primers for amplification of parts of the genomic sequence are those which bind to the underlined sequences of FIGS. 3 and/or 3a, or their complement, under conditions which will enable amplication. Preferred genomic primers for the amplification of all or part of exon 4 are as follows:

TABLE 1

| | Forward | |
|---|---|---|
| Amplicon | Primer | Reverse Primer |
| 4 | 4af | 4x |
| 4-1 (586 bp) | 4af | 4ar or 4ar inv/comp |
| 4-2 (323 bp) | 4b/cf | 4cr or 4cre inv/comp |
| 4-3 (628 bp) | 4df | 4b/dr or 4b/dr inv/comp |
| 4-4 | 4b/cf | 4b/dr |
| 4-1 + 4-2 + 4-3 | 4af | 4b/dr |
| 4-1 + 4-2 | 4af | 4cr or 4cr inv/comp |
| 4-2 + 4-3 | 4b/cf | 4b/dr |

Where primers:
4af is gaaacatcatgtatcttccatac
4x is gaaacatcatgtatcttccatacc
4cr is tccattccacaaagtagctg
4cr inv/comp is cagctactttgtggaatgga
4ar is aggcataaagaaatggagacg
4ar inv/comp is cgtctccatttctttatgcct
4df is catggaggaggtcgatgag
4b/4cf is gtcaccacctgcctccgtt
4b/4dr is cctgtatttctttctggtgccc
4b/dr inv/comp is gggcaccagaaagaaatacagg

Alternative primers are those described in Sale et al., Molecular Cancer therapeutics 2002. Use of these primers results in the amplicons shown in FIG. 3a.

Suitable primers for amplification of a β-tubulin transcript or cDNA include those which hybridise to the underlined sequences of FIG. 2, for example:

Forward: CTTGCCCCATACATACCTTGAG
Reverse: CCCAGACTGACCAAATACAAAG
Forward: GACCTGCAGCTGGACCGC
Reverse: TCAGGGTATTCTTCTCGGATCTT
Forward: TGGTTGATTCTGTCCTGGATG
Reverse: GTTGGTGTGGTCAGCCTCAGAG
Forward: TACAATGCCACCCTCTCCGT
Reverse: GCATCTGCTCATCGACCTCCT
Forward: GGAAGCCAGCAGTATCGAGC
Reverse: CTCACCGAAATCCTCCTCCTC
Forward: CTGAGAGCAACATGAACGACC
Reverse: GAGCGCCTACTATTGCCAG
Forward: GACCTCCGCAAGTTGGCAGT
Reverse: CAGGCAGCCATCATGTTCTTG

When amplifying the β-tubulin gene, it is preferable to do so in a manner which will reduce and preferable avoid altogether the concurrent amplification of pseudogenes. Suitable methods will be known to persons skilled in the art. A preferred method involves placing one amplication primer within an intron of the β-tubulin gene, preferably the intron between the third and fourth exons. Any suitable intron based primer may be used, and can be designed by a person skilled in the art using the primer design criteria disclosed herein, or methodology available in the art.

In many cases, it may be desirable either to analyse multiple variants in a sample from a single subject, or conversely to determine the presence or one or more variants in samples from a number of subjects. In such cases, it may be desirable to perform the analysis on a nucleic acid array, where either the probe primers or β-tubulin nucleic acid are immobilised on a support, either covalently or non-covalently. The arrays may be in any suitable form. Known arrays include wells, slides, chips, sheets, beads, fibres and membranes. The fabric of the array is preferably solid, for example paper, silicone, plastic, and glass. Attachment of the nucleic acid to the support may be mediated by any suitable method. Known methods include antibody-antigen interactions, streptavidin or avidin-biotin conjugates, hydrophobic interactions, UV cross linking, and chemical linkages.

Alternatively, the method of the invention may determine the presence of variants at positions 263, 293 and/or 294 of the β-tubulin protein as represented by FIG. 1. Again, any suitable method for determining variants in amino acid sequences may be used, including mass spectrometry (Verdiner-Pinard et al Biochemistry (2003) 42 5319-5357). Preferred methods include the use of antibodies or antibody fragments, or aptamers, which are capable of binding to, and discriminating between, different protein sequences.

A preferred antibody for use in the present invention is one which binds to the amino acid sequence. Antibodies can be made by the procedure set forth by standard procedures (Harlow and Lane, “Antibodies; A Laboratory Manual” Cold Spring Harbor Laboratory, Cold Spring Harbor, N.Y., 1998). Purified antigen can be injected into an animal in an amount and in intervals sufficient to elicit an immune response. Antibodies can either be purified directly, or spleen cells can be obtained from the animal. The cells are then fused with an immortal cell line and screened for antibody secretion. The antibodies can be used to screen DNA clone libraries for cells or phage, expressing or secreting antigen. Those positive clones can then be sequenced as described in, for example, Kellys et al., Bio/Technology 10:163-167 (1992) and Bebbington et al., Bio/Technology 10:169-175 (1992). The antibody is preferably specific for a variant amino acid sequence of the invention, and preferably, the antibody is sufficiently specific to distinguish between the reference β-tubulin protein and variants at the positions mentioned above. Preferably, the antigen being detected and/or used to generate a particular antibody will include the β-tubulin proteins or protein fragments as described herein.

Binding of antibodies, antibody fragments or aptamers may be detected by using appropriate methods, such as immunoflourescence assays, ELISA or immunoblotting. Preferably, a secondary antibody or ligand which binds to the first antibody may be used. The secondary antibody or ligand will be labelled, for example with a radiolabel, fluorescence or a suitable enzyme such as horse radish peroxidase.

The screening method for identifying suitable therapeutic agent is preferably carried out on cells, but may conceivably be conducted in an in vitro system comprising the appropriate β-tubulin nucleic acid and/or protein. The cells or in vitro system are exposed to a therapeutic agent being tested, by any suitable method. A suitable therapeutic agent is one which is able to stabilise or destabilise tubulin, thereby effecting the polymerisation and/or depolymerisation of β-tubulin. Such polymerisation and/or depolymerisation may be monitored by using any appropriate cellular marker associated therewith, for example proliferation of the cells (where cells are used).

The kits of the present invention will preferably comprise nucleic acid molecules, proteins, nucleic acid and/or protein binding agents as described herein, in the third to sixth aspects of the invention. Most preferably, the kit will comprise nucleic acid sequences able to hybridise to the β-tubulin gene, such as the probes or primers described herein, or protein binding agents specific for β-tubulin protein variants. The kit may additionally comprise a reference chart, detailing the correlation between the level of resistance or hypersensitivity to one or more therapeutic agents and the particular nucleotide or protein variants which are present.

The kit will also preferably comprise means for detection of the nucleic acid sequences or protein binding agents, and a substrate to which the nucleic acid sequences or protein binding agents are attached, for example an array as described herein.

The preferred embodiments of each aspect apply to the other aspects, mutatis mutandis.

A more complete understanding of the making and applications of the present invention can be obtained from the following example, which is provided for illustrative purposes only and is in no way limits the scope of the present invention.

### EXAMPLES

### Preparation of HM40 Type 1 β-Tubulin Regions for Mutational Analysis.

PCR products amplified from 5 control genomic DNA samples with the primers shown below were sequenced. The DNA sequences were then aligned with the AC006165 reference sequence, which indicated a 100% match across all four regions analyzed. The sequences were also aligned with the J00314 entry, which showed a few areas of discrepancy that could be related to sequencing or annotation errors in the J00314 entry. Cloning these PCR products and sequencing 15 clones representing each exon also gave identical results, indicating that the primers and conditions are only amplifying sequence from the AC006165 locus or a locus that contains regions that are 100% identical to AC006165.

Exon 1 primers:
cctctcctttctccctctc
and
tcttggcaggcacattt
Exon 2 primers:
ctgggacttgacctgttgt
and
cttccctgtctcccacttat
Exon 3 primers:
ccttcccttctgccagatttct
and
gaaacatcatgtatcttcca
Exon 4 primers:
As described in Table 1

Navigator Software (Transgenomic, Inc.) was used to analyze the HM40 sequence and design optimal amplicons for PCR and DHPLC analysis. The software uses proprietary algorithms to select amplicons based upon sequence content that defines the % helicity of a segment of DNA at a selected temperature and analysis conditions (mobile phase concentration, salt conc, length of amplicon, % GC) to produce melt profiles.

The primer sets of Table 1 and amplicons described herein were used in the HM40 exon 4 analysis. This includes PCR primers for DHPLC analysis and confirmatory DNA sequencing.

The sequences used to scan for mutations are exon containing fragments 38466 to 38775, 36246 to 36744, 35818 to 36250 35256 to 35841 and 34467 to 35354 of AC006161, and FIG. 1. PCR primers for each fragment are from the provided reference article (Sale, et. al., Molecular Cancer Therapeutics, 2002). Exon 4 may need to be divided up into 3-4 fragments instead of two as described in the article. The region for amplification may be expanded for better primer design if needed.

### Beta Tubulin Exon 4 Inverted Intronic-Anchored MASA Assay Design

DNA extracted from a slide, 5-10 μm thick. 1 to 5 μl of the DNA prep was used for a 50 μl PCR reaction. Primers were used at a concentration of 10 mM. Hotmaster Taq or AmpliTaq Gold was used as the DNA Polymerase. Annealing temps for the MASA were empirically determined using a positive and negative control. The optimal annealing temp is one that obtains a robust single from the positive control and an undetectable signal from the negative control. A gradient thermal cycler was used to select the optimal annealing temps and for the MASA of test samples.

5 μl of the MASA PCR product was injected for dsDNA sizing analysis of the WAVE-HS system, which can detect 5-10 pg of dsDNA

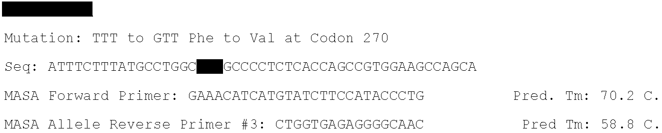

PCR Conditions:

| Step | Temp | Time |
|---|---|---|
| 1 | 95 C. | 2 minutes |
| 2 | 95 C. | 15 seconds |
| 3 | 62.5 C. | 15 seconds |
| 4 | 68 C. | 15 seconds |
| 5 | Go to step 2 39 times | |
| 6 | 68 C. | 5 minutes |
| 7 | 4 C. | Hold |

Polymerase: Hotmaster (Eppendorf).
Note:
This assay was developed using for use with approximately 10 ng of gDNA. More DNA may cause increased
background and false positives.

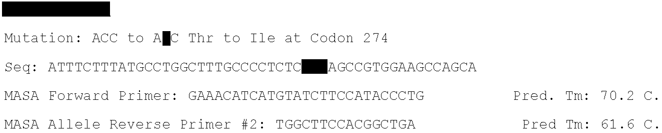

PCR Conditions:

| Step | Temp | Time |
|---|---|---|
| 1 | 95 C. | 2 minutes |
| 2 | 95 C. | 15 seconds |
| 3 | 63.5 C. | 15 seconds |
| 4 | 68 C. | 15 seconds |
| 5 | Go to step 2 39 times | |
| 6 | 68 C. | 5 minutes |
| 7 | 4 C. | Hold |

Polymerase: Hotmaster (Eppendorf).
Note:
This assay was developed using for use with approximately 10 ng of gDNA. More DNA may cause increased
background and false positives.

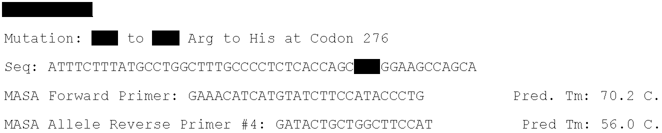

PCR Conditions:

| Step | Temp | Time |
|---|---|---|
| 1 | 95 C. | 2 minutes |
| 2 | 95 C. | 15 seconds |
| 3 | 62 C. | 15 seconds |
| 4 | 68 C. | 15 seconds |
| 5 | Go to step 2 39 times | |
| 6 | 68 C. | 5 minutes |
| 7 | 4 C. | Hold |

Polymerase: Hotmaster (Eppendorf).
Note:
This assay was developed using for use with approximately 10 ng of gDNA. More DNA may cause increased
background and false positives.

Primers used in HM-40 beta-tubulin mutant allele specific amplification (MASA) using a 5′ forward intronic-anchored primer and a 3′ mutant allele specific primer

### Beta Tubulin Exon 4 MASA Assay Primers

Beta Tubulin Exon 4

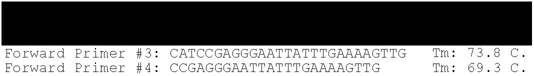

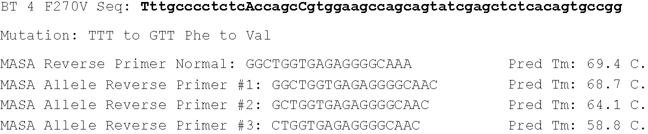

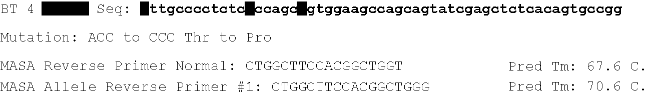

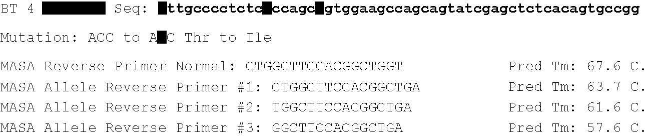

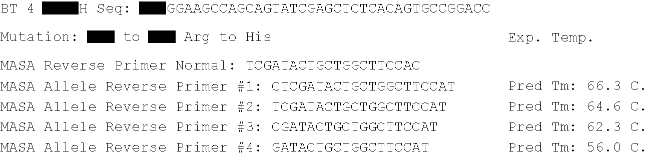

BT cDNA Analysis

Primers and conditions used for RT-PCR of HM-40 beta-tubulin transcripts

| | |
|---|---|
| BT cDNA Primers: | |
| BT Seq1 For: | CTTGCCCCATACATACCTTGAG |
| BT Seq1 Rev: | CCCAGACTGACCAAATACAAAG |
| BT Seq2 For: | GACCTGCAGCTGGACCGC |
| BT Seq2 Rev: | TCAGGGTATTCTTCTCGGATCTT |
| BT Seq3 For: | TGGTTGATTCTGTCCTGGATG |
| BT Seq3 Rev: | GTTGGTGTGGTCAGCCTCAGAG |
| BT Seq4 For: | TACAATGCCACCCTCTCCGT |
| BT Seq4 Rev: | GCATCTGCTCATCGACCTCCT |
| BT Seq5 For: | GGAAGCCAGCAGTATCGAGC |
| BT Seq5 Rev: | CTCACCGAAATCCTCCTCCTC |
| BT Seq6 For: | CTGAGAGCAACATGAACGACC |
| BT Seq6 Rev: | GAGCGCCTACTATTGCCAG |
| BT HZ Seq For: | GACCTCCGCAAGTTGGCAGT |
| BT HZ Seq Rev: | CAGGCAGCCATCATGTTCTTG |

RT reaction performed using Ambion's Retroscript First Strand Synthesis Kit (Item # 1710) as per the manufacturers suggestion using random decamers included in this kit.

Template—2 microliters of cDNA reaction as described above.

PCR Conditions:

| Step | Temp | Time |
|---|---|---|
| 1 | 95 C. | 2 minutes |
| 2 | 95 C. | 15 seconds |
| 3 | 56 C. | 15 seconds |
| 4 | 68 C. | 15 seconds |
| 5 | Go to step 2 | 39 times |
| 6 | 68 C. | 5 minutes |
| 7 | 4 C. | Hold |

Polymerase: Hotmaster (Eppendorf).

Beta Tubulin Exon 4 MASA Primers (cDNA or gDNA analysis)-non-intron anchored
MASA

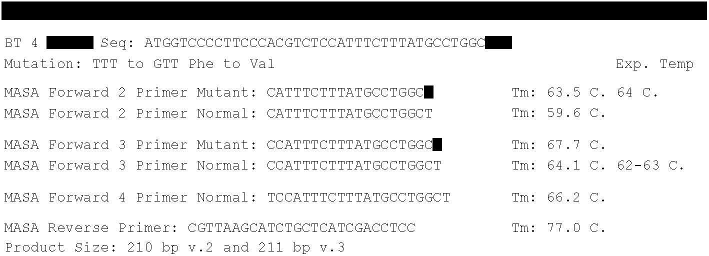

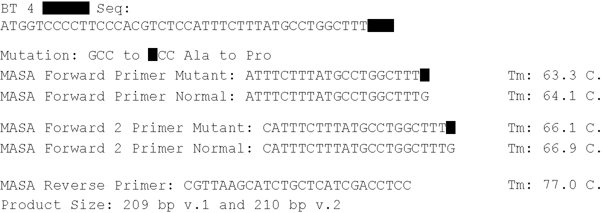

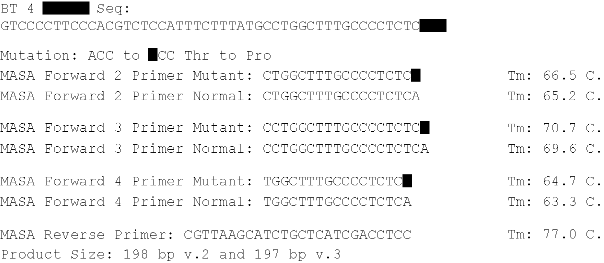

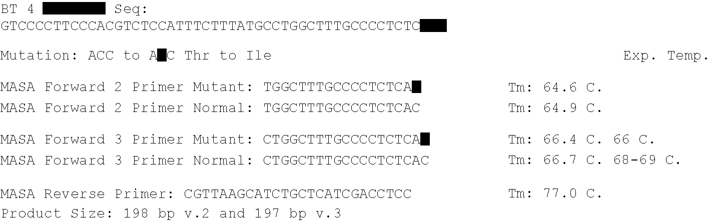

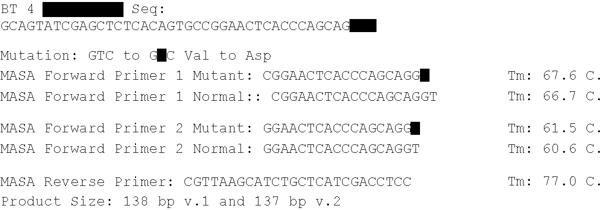

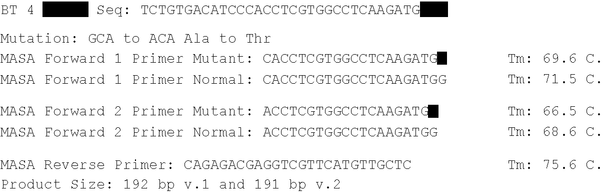

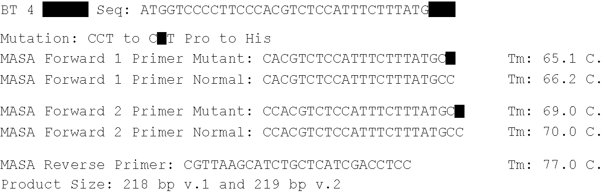

1. A method of determining the potential of a subject to respond to a therapeutic agent, the method comprising determining the presence of an amino acid variant at one or more of positions 263, 268, 270, 274, 276, 293, and 364 of the β-tubulin protein of the subject, as represented in FIG. 1, wherein the presence or absence of a variant is indicative of resistance or hypersensitivity to the therapeutic agent.

2. A method according to claim 1, comprising determining the presence of a nucleotide variant in a nucleic acid sequence which codes for β-tubulin which causes an amino acid variation at positions 263, 268, 270, 274, 276, 293 and/or 364 of the β-tubulin protein as represented by FIG. 1.

3. A method according to claim 2 comprising determining the presence of a nucleotide variant at one or more of positions 787, 803, 808, 821, 827, 878 and/or 1091 of the nucleic acid sequence encoding β-tubulin, as represented by FIG. 2.

4. A method according to claim 1, comprising first establishing the presence of variants in the β-tubulin protein or gene, as represented by FIGS. 1 and 2 respectively.

5. A method according to claim 4, wherein the presence of variants is established by heteroduplex analysis of a nucleic acid sequence encoding β-tubulin, or fragments thereof.

6. A method according to claim 4, wherein the presence of variants is established by DHPLC, mismatch detection, gel electrophoresis or SSCP.

7. A method according to claim 6, comprising the additional steps of:
(a) separating the strands of the nucleic acid sample to be tested;
(b) forming a duplex with a strand known not to contains any variant nucleotides at one or more of positions 787, 803, 808, 820, 821, 878 and/or 1091 of FIG. 2;
(c) producing a melting profile for the duplex, wherein profile indicates the presence of one or more variants in the strand being tested.

8. A method according to claim 2, wherein the nucleic acid sequence encoding β-tubulin is amplified prior to determining the presence of a nucleotide variant.

9. A method according to claim 2, wherein the presence of a nucleotide variant is determined by sequencing of PCR products, direct probing direct cloning and sequencing, Allele Specific Hybridisation, Mutant Allele Specific Amplification (MASA), RFLP, polymerase mediated primer extension, single base extension, or rolling circle amplification following allele specific ligation

10. A method according to claim 9, wherein the presence of a nucleotide variant is determined by MASA.

11. A method according to claim 10, wherein a primer specific for the wild type, or reference, allele is additionally used, said primer being unable to mediate amplification.

12. A method according to claim 1 wherein the presence of an amino acid variant is determined by mass spectrometry, or by the use of protein binding agents.

13. A method according to claim 1, wherein the therapeutic agent is one which targets, or mediates its action via. β-tubulin.

14. A method according to claim 13, wherein the therapeutic agent is for use in the treatment of a disease which results from de-regulation of a process involving β-tubulin or microtubules.

15. A method of treating disease in a subject, the method comprising (a) determining the potential of a subject to respond to a therapeutic agent according to claim 1, wherein the presence or absence of a nucleotide or amino acid variant is indicative of resistance or hypersensitivity to the therapeutic agent; and (b) administering a therapeutic agent to which the subject is not resistant.

16. An isolated or recombinant nucleic acid molecule comprising a nucleic acid sequence encoding β-tubulin, or a nucleic acid sequence complementary thereto, having a nucleotide variant which causes a variant at position 263, 268, 276 and/or 293 of the β-tubulin protein as represented by FIG. 1; and fragments thereof.

17. An isolated or recombinant nucleic acid molecule according to claim 5, wherein the nucleotide variant is present at a position corresponding to 787, 803 827, and/or 878, of the nucleic acid sequence represented by FIG. 2; and fragments thereof.

18. An isolated or recombinant nucleic acid molecule nucleic acid molecule comprising a nucleic acid sequence which is able to hybridise to a nucleic acid sequence encoding β-tubulin as represented in FIG. 2, or to a strand complementary thereto.

19. An isolated or recombinant nucleic acid molecule according to claim 7 which is able to hybridise to a nucleotide variant which causes a variation at positions 263, 268, 276 and/or 293 of the β-tubulin protein represented by FIG. 1, or to a region flanking such a variant.

20. An isolated β-tubulin protein as represented by FIG. 1, having an amino acid substitution at position 263, 268, 276 and/or 293.

21. Protein binding agents specific for a variant at positions 263, 268, 276 and/or 293 of the β-tubulin protein represented by FIG. 1.

22. A method of screening for suitable therapeutic agents, comprising
a) providing cells comprising an amino acid variant at one or more of positions 263, 268, 270, 274, 276, 293 and 364 of the β-tubulin protein as represented by FIG. 1; or nucleic acid sequences encoding β-tubulin and having a variant which cause one or more of the amino acid variants; and
b) exposing the cells to a therapeutic agent; and
c) monitoring the polymerisation and/or depolymerisation of β-tubulin in the cells upon exposure to the therapeutic agent; wherein suitable therapeutic agents are those which are capable of affecting the polymerisation and/or depolymerisation of β-tubulin in the cells.

23. A method according to claim 22 wherein monitoring the polymerisation and/or depolymerisation is performed by monitoring proliferation of the cells.

24. A kit comprising nucleic acid sequences according to claim 16, and/or protein binding agents, for determining the presence of a variant at one or more of positions of β-tubulin 263, 268, and/or 276, 293 of the β-tubulin protein as represented by FIG. 1, together with a reference chart detailing the correlation between nucleotide or amino acid variants and resistance of the subject to therapeutic agents.


SEQUENCE LISTING (170 sequences)

SEQ ID NO: 1 - DNA, 23 bp, Artificial
  Primer
gaaacatcatgtatcttccatac

SEQ ID NO: 2 - DNA, 24 bp, Artificial
  Primer
gaaacatcatgtatcttccatacc

SEQ ID NO: 3 - DNA, 20 bp, Artificial
  Primer
tccattccacaaagtagctg

SEQ ID NO: 4 - DNA, 20 bp, Artificial
  Primer
cagctactttgtggaatgga

SEQ ID NO: 5 - DNA, 21 bp, Artificial
  Primer
aggcataaagaaatggagacg

SEQ ID NO: 6 - DNA, 21 bp, Artificial
  Primer
cgtctccatttctttatgcct

SEQ ID NO: 7 - DNA, 19 bp, Artificial
  Primer
catggaggaggtcgatgag

SEQ ID NO: 8 - DNA, 19 bp, Artificial
  Primer
gtcaccacctgcctccgtt

SEQ ID NO: 9 - DNA, 22 bp, Artificial
  Primer
cctgtatttctttctggtgccc

SEQ ID NO: 10 - DNA, 22 bp, Artificial
  Primer
gggcaccagaaagaaatacagg

SEQ ID NO: 11 - DNA, 22 bp, Artificial
  Primer
cttgccccatacataccttgag

SEQ ID NO: 12 - DNA, 22 bp, Artificial
  Primer
cccagactgaccaaatacaaag

SEQ ID NO: 13 - DNA, 18 bp, Artificial
  Primer
gacctgcagctggaccgc

SEQ ID NO: 14 - DNA, 23 bp, Artificial
  Primer
tcagggtattcttctcggatctt

SEQ ID NO: 15 - DNA, 21 bp, Artificial
  Primer
tggttgattctgtcctggatg

SEQ ID NO: 16 - DNA, 22 bp, Artificial
  Primer
gttggtgtggtcagcctcagag

SEQ ID NO: 17 - DNA, 20 bp, Artificial
  Primer
tacaatgccaccctctccgt

SEQ ID NO: 18 - DNA, 21 bp, Artificial
  Primer
gcatctgctcatcgacctcct

SEQ ID NO: 19 - DNA, 20 bp, Artificial
  Primer
ggaagccagcagtatcgagc

SEQ ID NO: 20 - DNA, 21 bp, Artificial
  Primer
ctcaccgaaatcctcctcctc

SEQ ID NO: 21 - DNA, 21 bp, Artificial
  Primer
ctgagagcaacatgaacgacc

SEQ ID NO: 22 - DNA, 19 bp, Artificial
  Primer
gagcgcctactattgccag

SEQ ID NO: 23 - DNA, 20 bp, Artificial
  Primer
gacctccgcaagttggcagt

SEQ ID NO: 24 - DNA, 21 bp, Artificial
  Primer
caggcagccatcatgttcttg

SEQ ID NO: 25 - DNA, 19 bp, Artificial
  Primer
cctctcctttctccctctc

SEQ ID NO: 26 - DNA, 17 bp, Artificial
  Primer
tcttggcaggcacattt

SEQ ID NO: 27 - DNA, 19 bp, Artificial
  Primer
ctgggacttgacctgttgt

SEQ ID NO: 28 - DNA, 20 bp, Artificial
  Primer
cttccctgtctcccacttat

SEQ ID NO: 29 - DNA, 22 bp, Artificial
  Primer
ccttcccttctgccagatttct

SEQ ID NO: 30 - DNA, 20 bp, Artificial
  Primer
gaaacatcatgtatcttcca

SEQ ID NO: 31 - DNA, 49 bp, Homo sapiens
  misc_feature: BT 4 F270V
atttctttatgcctggctttgcccctctcaccagccgtggaagccagca

SEQ ID NO: 32 - DNA, 49 bp, Artificial
  Mutated BT 4 F270V
atttctttatgcctggcgttgcccctctcaccagccgtggaagccagca

SEQ ID NO: 33 - DNA, 27 bp, Artificial
  Primer
gaaacatcatgtatcttccataccctg

SEQ ID NO: 34 - DNA, 17 bp, Artificial
  Primer
ctggtgagaggggcaac

SEQ ID NO: 35 - DNA, 49 bp, Homo sapiens; identical to SEQ ID NO: 31
  misc_feature: BT 4 R276H

SEQ ID NO: 36 - DNA, 49 bp, Artificial
  Mutated BT 4 T274Ile
atttctttatgcctggctttgcccctctcatcagccgtggaagccagca

SEQ ID NO: 37 - DNA, 27 bp, Artificial
  Primer
gaaacatcatgtatcttccataccctg

SEQ ID NO: 38 - DNA, 16 bp, Artificial
  Primer
tggcttccacggctga

SEQ ID NO: 39 - DNA, 49 bp, Homo sapiens; identical to SEQ ID NO: 31
  misc_feature: BT 4 R276H

SEQ ID NO: 40 - DNA, 49 bp, Artificial
  Mutated BT 4 R276H
atttctttatgcctggctttgcccctctcaccagccatggaagccagca

SEQ ID NO: 41 - DNA, 27 bp, Artificial
  Primer
gaaacatcatgtatcttccataccctg

SEQ ID NO: 42 - DNA, 18 bp, Artificial
  Primer
gatactgctggcttccat

SEQ ID NO: 43 - DNA, 27 bp, Artificial
  Primer
gaaacatcatgtatcttccataccctg

SEQ ID NO: 44 - DNA, 26 bp, Artificial
  Primer
gagcttttctcctgactgcattccag

SEQ ID NO: 45 - DNA, 27 bp, Artificial
  Primer
catccgagggaattatttgaaaagttg

SEQ ID NO: 46 - DNA, 24 bp, Artificial
  Primer
ccgagggaattatttgaaaagttg

SEQ ID NO: 47 - DNA, 55 bp, Homo sapiens
  misc_feature: BT 4 F270V
tttgcccctctcaccagccgtggaagccagcagtatcgagctctcacagtgccgg

SEQ ID NO: 48 - DNA, 55 bp, Artificial
  Mutated BT 4 F270V
gttgcccctctcaccagccgtggaagccagcagtatcgagctctcacagtgccgg

SEQ ID NO: 49 - DNA, 19 bp, Artificial
  Primer
ggctggtgagaggggcaaa

SEQ ID NO: 50 - DNA, 19 bp, Artificial
  Primer
ggctggtgagaggggcaac

SEQ ID NO: 51 - DNA, 18 bp, Artificial
  Primer
gctggtgagaggggcaac

SEQ ID NO: 52 - DNA, 17 bp, Artificial
  Primer
ctggtgagaggggcaac

SEQ ID NO: 53 - DNA, 55 bp, Homo sapiens; identical to SEQ ID NO: 47
  misc_feature: BT 4 T274P

SEQ ID NO: 54 - DNA, 55 bp, Artificial
  Mutated BT 4 T274P
tttgcccctctccccagccgtggaagccagcagtatcgagctctcacagtgccgg

SEQ ID NO: 55 - DNA, 18 bp, Artificial
  Primer
ctggcttccacggctggt

SEQ ID NO: 56 - DNA, 18 bp, Artificial
  Primer
ctggcttccacggctggg

SEQ ID NO: 57 - DNA, 55 bp, Homo sapiens; identical to SEQ ID NO: 47
  misc_feature: BT 4 T274I1e

SEQ ID NO: 58 - DNA, 55 bp, Artificial
  Mutated BT 4 T274I1e
tttgcccctctcatcagccgtggaagccagcagtatcgagctctcacagtgccgg

SEQ ID NO: 59 - DNA, 18 bp, Artificial
  Primer
ctggcttccacggctggt

SEQ ID NO: 60 - DNA, 17 bp, Artificial
  Primer
ctggcttccacggctga

SEQ ID NO: 61 - DNA, 16 bp, Artificial
  Primer
tggcttccacggctga

SEQ ID NO: 62 - DNA, 15 bp, Artificial
  Primer
ggcttccacggctga

SEQ ID NO: 63 - DNA, 40 bp, Homo sapiens
  misc_feature: BT 4 R276H
cgtggaagccagcagtatcgagctctcacagtgccggacc

SEQ ID NO: 64 - DNA, 40 bp, Artificial
  Mutated BT 4 R276H
catggaagccagcagtatcgagctctcacagtgccggacc

SEQ ID NO: 65 - DNA, 20 bp, Artificial
  Primer
tcgatactgctggcttccac

SEQ ID NO: 66 - DNA, 21 bp, Artificial
  Primer
ctcgatactgctggcttccat

SEQ ID NO: 67 - DNA, 20 bp, Artificial
  Primer
tcgatactgctggcttccat

SEQ ID NO: 68 - DNA, 19 bp, Artificial
  Primer
cgatactgctggcttccat

SEQ ID NO: 69 - DNA, 18 bp, Artificial
  Primer
gatactgctggcttccat

SEQ ID NO: 70 - DNA, 22 bp, Artificial
  Primer
cttgccccatacataccttgag

SEQ ID NO: 71 - DNA, 22 bp, Artificial
  Primer
cccagactgaccaaatacaaag

SEQ ID NO: 72 - DNA, 18 bp, Artificial
  Primer
gacctgcagctggaccgc

SEQ ID NO: 73 - DNA, 23 bp, Artificial
  Primer
tcagggtattcttctcggatctt

SEQ ID NO: 74 - DNA, 21 bp, Artificial
  Primer
tggttgattctgtcctggatg

SEQ ID NO: 75 - DNA, 22 bp, Artificial
  Primer
gttggtgtggtcagcctcagag

SEQ ID NO: 76 - DNA, 20 bp, Artificial
  Primer
tacaatgccaccctctccgt

SEQ ID NO: 77 - DNA, 21 bp, Artificial
  Primer
gcatctgctcatcgacctcct

SEQ ID NO: 78 - DNA, 20 bp, Artificial
  Primer
ggaagccagcagtatcgagc

SEQ ID NO: 79 - DNA, 21 bp, Artificial
  Primer
ctcaccgaaatcctcctcctc

SEQ ID NO: 80 - DNA, 21 bp, Artificial
  Primer
ctgagagcaacatgaacgacc

SEQ ID NO: 81 - DNA, 19 bp, Artificial
  Primer
gagcgcctactattgccag

SEQ ID NO: 82 - DNA, 20 bp, Artificial
  Primer
gacctccgcaagttggcagt

SEQ ID NO: 83 - DNA, 21 bp, Artificial
  Primer
caggcagccatcatgttcttg

SEQ ID NO: 84 - DNA, 42 bp, Homo sapiens
  misc_feature: BT 4 F270V
atggtccccttcccacgtctccatttctttatgcctggcttt

SEQ ID NO: 85 - DNA, 42 bp, Artificial
  Mutated BT 4 F270V
atggtccccttcccacgtctccatttctttatgcctggcgtt

SEQ ID NO: 86 - DNA, 19 bp, Artificial
  Primer
catttctttatgcctggcg

SEQ ID NO: 87 - DNA, 19 bp, Artificial
  Primer
catttctttatgcctggct

SEQ ID NO: 88 - DNA, 20 bp, Artificial
  Primer
ccatttctttatgcctggcg

SEQ ID NO: 89 - DNA, 20 bp, Artificial
  Primer
ccatttctttatgcctggct

SEQ ID NO: 90 - DNA, 21 bp, Artificial
  Primer
tccatttctttatgcctggct

SEQ ID NO: 91 - DNA, 26 bp, Artificial
  Primer
cgttaagcatctgctcatcgacctcc

SEQ ID NO: 92 - DNA, 45 bp, Homo sapiens
  misc_feature: BT 4 A271P
atggtccccttcccacgtctccatttctttatgcctggctttgcc

SEQ ID NO: 93 - DNA, 45 bp, Artificial
  Mutated BT 4 A271P
atggtccccttcccacgtctccatttctttatgcctggctttccc

SEQ ID NO: 94 - DNA, 21 bp, Artificial
  Primer
atttctttatgcctggctttc

SEQ ID NO: 95 - DNA, 21 bp, Artificial
  Primer
atttctttatgcctggctttg

SEQ ID NO: 96 - DNA, 22 bp, Artificial
  Primer
catttctttatgcctggctttc

SEQ ID NO: 97 - DNA, 22 bp, Artificial
  Primer
catttctttatgcctggctttg

SEQ ID NO: 98 - DNA, 26 bp, Artificial
  Primer
cgttaagcatctgctcatcgacctcc

SEQ ID NO: 99 - DNA, 51 bp, Homo sapiens
  misc_feature: BT 4 T274P
gtccccttcccacgtctccatttctttatgcctggctttgcccctctcacc

SEQ ID NO: 100 - DNA, 51 bp, Artificial
  BT 4 T274P
gtccccttcccacgtctccatttctttatgcctggctttgcccctctcccc

SEQ ID NO: 101 - DNA, 18 bp, Artificial
  Primer
ctggctttgcccctctcc

SEQ ID NO: 102 - DNA, 18 bp, Artificial
  Primer
ctggctttgcccctctca

SEQ ID NO: 103 - DNA, 19 bp, Artificial
  Primer
cctggctttgcccctctcc

SEQ ID NO: 104 - DNA, 19 bp, Artificial
  Primer
cctggctttgcccctctca

SEQ ID NO: 105 - DNA, 17 bp, Artificial
  Primer
tggctttgcccctctcc

SEQ ID NO: 106 - DNA, 17 bp, Artificial
  Primer
tggctttgcccctctca

SEQ ID NO: 107 - DNA, 26 bp, Artificial
  Primer
cgttaagcatctgctcatcgacctcc

SEQ ID NO: 108 - DNA, 51 bp, Homo sapiens; identical to SEQ ID NO: 99
  misc_feature: BT 4 T274Ile

SEQ ID NO: 109 - DNA, 51 bp, Artificial
  Mutated BT 4 T274Ile
gtccccttcccacgtctccatttctttatgcctggctttgcccctctcatc

SEQ ID NO: 110 - DNA, 18 bp, Artificial
  Primer
tggctttgcccctctcat

SEQ ID NO: 111 - DNA, 18 bp, Artificial
  Primer
tggctttgcccctctcac

SEQ ID NO: 112 - DNA, 19 bp, Artificial
  Primer
ctggctttgcccctctcat

SEQ ID NO: 113 - DNA, 19 bp, Artificial
  Primer
ctggctttgcccctctcac

SEQ ID NO: 114 - DNA, 26 bp, Artificial
  Primer
cgttaagcatctgctcatcgacctcc

SEQ ID NO: 115 - DNA, 43 bp, Homo sapiens
  misc_feature: BT 4 Val293Asp
gcagtatcgagctctcacagtgccggaactcacccagcaggtc

SEQ ID NO: 116 - DNA, 43 bp, Artificial
  Mutated BT 4 T274Ile
gcagtatcgagctctcacagtgccggaactcacccagcaggac

SEQ ID NO: 117 - DNA, 19 bp, Artificial
  Primer
cggaactcacccagcagga

SEQ ID NO: 118 - DNA, 19 bp, Artificial
  Primer
cggaactcacccagcaggt

SEQ ID NO: 119 - DNA, 18 bp, Artificial
  Primer
ggaactcacccagcagga

SEQ ID NO: 120 - DNA, 18 bp, Artificial
  Primer
ggaactcacccagcaggt

SEQ ID NO: 121 - DNA, 26 bp, Artificial
  Primer
cgttaagcatctgctcatcgacctcc

SEQ ID NO: 122 - DNA, 35 bp, Homo sapiens
  misc_feature: BT 4 A364T
tctgtgacatcccacctcgtggcctcaagatggca

SEQ ID NO: 123 - DNA, 35 bp, Artificial
  Mutated BT 4 A364T
tctgtgacatcccacctcgtggcctcaagatgaca

SEQ ID NO: 124 - DNA, 21 bp, Artificial
  Primer
cacctcgtggcctcaagatga

SEQ ID NO: 125 - DNA, 21 bp, Artificial
  Primer
cacctcgtggcctcaagatgg

SEQ ID NO: 126 - DNA, 20 bp, Artificial
  Primer
acctcgtggcctcaagatga

SEQ ID NO: 127 - DNA, 20 bp, Artificial
  Primer
acctcgtggcctcaagatgg

SEQ ID NO: 128 - DNA, 26 bp, Artificial
  Primer
cagagacgaggtcgttcatgttgctc

SEQ ID NO: 129 - DNA, 36 bp, Homo sapiens
  misc_feature: BT 4 P268H
atggtccccttcccacgtctccatttctttatgcct

SEQ ID NO: 130 - DNA, 36 bp, Artificial
  BT 4 P268H
atggtccccttcccacgtctccatttctttatgcat

SEQ ID NO: 131 - DNA, 22 bp, Artificial
  Primer
cacgtctccatttctttatgca

SEQ ID NO: 132 - DNA, 22 bp, Artificial
  Primer
cacgtctccatttctttatgcc

SEQ ID NO: 133 - DNA, 23 bp, Artificial
  Primer
ccacgtctccatttctttatgca

SEQ ID NO: 134 - DNA, 23 bp, Artificial
  Primer
ccacgtctccatttctttatgcc

SEQ ID NO: 135 - DNA, 26 bp, Artificial
  Primer
cgttaagcatctgctcatcgacctcc

SEQ ID NO: 136 - protein, 444 aa, Homo sapiens [sequence omitted]

SEQ ID NO: 137 - DNA, 1954 bp, Homo sapiens
ctccctctcagaaccttcctgccgtcgcgtttgcacctcgctgctccagcctctggggcgcattccaaccttccagcctgcgacctgcggagaaaaaaaattacttattttcttgccccatacataccttgaggcgagcaaaaaaattaaattttaaccatgagggaaatcgtgcacatccaggctggtcaatgtggcaaccagatcggtgccaagttctgggaggtgatcagtgatgaacatggcatcgaccccaccggcacctaccacggggacagcgacctgcagctggaccgcatctctgtgtactacaatgaagccacaggtggcaaatatgttcctcgtgccatcctggtggatctagaacctgggaccatggactctgttcgctcaggtccttttggccagatctttagaccagacaactttgtatttggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaggctgaccacaccaacctacggggatctgaaccaccttgtctcaggcaccatggagtgtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggacctcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggagagcccccatcacctcaggcttctcagttcccttagccgtcttactcaactgcccctttcctctccctcagaatttgtgtttgctgcctctatcttgttttttgttttttcttctggggggggtctagaattccagaacagtgcctggcaatagtaggcgctcaataaatacttgtttgttgaatctctcctctctctttccactctgggaaacctaggtttctgccattctgggtgaccctgtatttctttctggtgcccattccatttgtccagttaatattcctcttaaaaatctccaagaagtgggtctccagatcccatttagaaccaaccaggtgctgaaaacattagtagataattggccatcatcctaagccaaagtagaaaatggtagaaggtagtgggtagagtcactatataggaaggggatgggattttcccattctaaagtttt

SEQ ID NO: 138 - DNA, 427 bp, Homo sapiens
cctctcctttctccctctcagaaccttcctgccgtcgcgtttgcacctcgctgctccagcctctggggcgcattccaaccttccagcctgcgacctgcggagaaaaaaaattacttattttcttgccccatacataccttgaggcgagcaaaaaaattaaattttaaccatgagggaaatcgtgcacatccaggctggtcagtgtggcaaccagatcggtgccaaggtaagaattttacacctcttttatttctttttacaaggaaaaatccaggtaagttatgaaaaaatggttgtggggcatttgcacccgctatccttaatcaagatttgcccctctcaagtttgttacatttatatatataacaattgtagctagcatttgcctttggaaagctgggaatcatttttcttggcaggcacattt

SEQ ID NO: 139 - DNA, 204 bp, Homo sapiens
ctgggacttgacctgttgtggtctcgttgctccccctcggcagttctgggaggtgatcagtgatgaacatggcatcgaccccaccggcacctaccacggggacagcgacctgcagctggaccgcatctctgtgtactacaatgaagccacaggtaagggcaggagcccgggcagctcaggttcccttccctgtctcccacttat

SEQ ID NO: 140 - DNA, 430 bp, Homo sapiens
ccttcccttctgccagatttcacagctcttaactttattctctgtaggtggcaaatatgttcctcgtgccatcctggtggatctagaacctgggaccatggactctgttcgctcaggtccttttggccagatctttagaccagacaactttgtatttggtgagttatacagatgatattagcagatgatataccatcgtgttcaacttatttgggtgcaaggacacagcaaaagttaggagatgattgttgtattggagtgctaatacagaaatgtgttctgaaatctaacggagggtagaggtagtgcctactattgctggtaaattatggggcagtagggggagaatatatcacagtgaaggagaaagaagatacatccgagggaattatttgaaaagttgaaagatggaaacatcatgtatcttcca

SEQ ID NO: 141 - DNA, 1375 bp, Homo sapiens
gaaacatcatgtatcttccataccctgttaattgagcttttctcctgactgcattccaggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaatgtctcctctctctttccactctgggaaacctaggtttctgccattctgggtgaccctgtatttctttctggtgccc

SEQ ID NO: 142 - DNA, 586 bp, Homo sapiens
gaaacatcatgtatcttccataccctgttaattgagcttttctcctgactgcattccaggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcct

SEQ ID NO: 143 - DNA, 328 bp, Homo sapiens
gtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatgga

SEQ ID NO: 144 - DNA, 628 bp, Homo sapiens
catgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaatgtctcctctctctttccactctgggaaacctaggtttctgccattctgggtgaccctgtatttctttctggtgccc

SEQ ID NO: 145 - DNA, 1375 bp, Homo sapiens; identical to SEQ ID NO: 141

SEQ ID NO: 146 - DNA, 810 bp, Homo sapiens
gaaacatcatgtatcttccataccctgttaattgagcttttctcctgactgcattccaggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtgga

SEQ ID NO: 147 - DNA, 586 bp, Homo sapiens; identical to SEQ ID NO: 142

SEQ ID NO: 148 - DNA, 657 bp, Homo sapiens
gtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaatgtctcctctctctttccactctgggaaacctaggtttctgccattctgggtgaccctgtatttctttctggtgccc

SEQ ID NO: 149 - DNA, 888 bp, Homo sapiens
gtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggtctagaacagtgcctggcacatagtaggcgctcaataaatacttgtttgttgaatgtctcctctctctttccactctgggaaacctaggtttctgccattctgggtgaccctgtatttctttctggtgccc

SEQ ID NO: 150 - DNA, 310 bp, Homo sapiens
ggatagcgggtgcaaatgccccacaaccattttttcataacttacctggatttttccttgtaaaaagaaataaaagaggtgtaaaattcttaccttggcaccgatctggttgccacactgaccagcctggatgtgcacgatttccctcatggttaaaatttaatttttttgctcgcctcaaggtatgtatggggcaagaaaataagtaattttttttctccgcaggtcgcaggctggaaggttggaatgcgccccagaggctggagcagcgaggtgcaaacgcgacggcaggaaggttctgagagggaga

SEQ ID NO: 151 - DNA, 501 bp, Homo sapiens
ggaaggtttatagatatactggaaatgggagacagcagggatcagagacttgtcattccaggtcccgccaccaggtggcagcagacgtctttggccccgacggtggttcacgaaagggacaaaatgacagattcacccaaaggggataaggcgtgcccagaaatggaaagagatcccagataagtgggagacagggaagggaacctgagctgcccgggctcctgcccttacctgtggcttcattgtagtacacagagatgcggtccagctgcaggtcgctgtccccgtggtaggtgccggtggggtcgatgccatgttcatcactgatcacctcccagaactgccgagggggagcaacgagaccacaacaggtcaagtcccagccaactatgtccccaactaccattttatttcatctttttcttaattttttttgagacggagtctcgctctgtcaccaggctggagtgcagtggcgctatctcggctcactgcaacctc

SEQ ID NO: 152 - DNA, 433 bp, Homo sapiens
ggtatggaagatacatgatgtttccatctttcaacttttcaaataattccctcggatgtatcttctttctccttcactgtgatatattctccccctactgccccataatttaccagcaatagtaggcactacctctaccctccgttagatttcagaacacatttctgtattagcactccaatacaacaatcatctcctaacttttgctgtgtccttgcacccaaataagttgaacacgatggtatatcatctgctaatatcatctgtataactcaccaaatacaaagttgtctggtctaaagatctggccaaaaggacctgagcgaacagagtccatggtcccaggttctagatccaccaggatggcacgaggaacatatttgccacctacagagaataaagttaagagctgtgaaatctggcagaagggaag

SEQ ID NO: 153 - DNA, 586 bp, Homo sapiens
aggcataaagaaatggagacgtgggaaggggaccatgttgactgccaacttgcggaggtcagcattgagctggccagggaaacggaggcaggtggtgacaccactcatggtggctgagacaaggtggttcagatccccgtaggttggtgtggtcagcttcagagtgcggaagcagatatcatagagggcctcgttgtcaatgcaataggtctcatcagtattctctaccaactgatggacggagagggtggcattgtagggctcgaccacggtgtcagacactttgggtgaaggcaccacactgaaggtattcatgatgcgatcagggtattcttctcggatcttgctgataaggagagtgcccattccagagcctgtgcccccgcccagtgagtgggtcagctggaagccctgcaggcagtcacagctctctgcctccttccgtaccacatccaggacagaatcaaccagctcggcgccctctgtgtagtggcctttggcccagttgttacctgccccagactgacctggaatgcagtcaggagaaaagctcaattaacagggtatggaagatacatgatgtttc

SEQ ID NO: 154 - DNA, 888 bp, Homo sapiens
gggcaccagaaagaaatacagggtcacccagaatggcagaaacctaggtttcccagagtggaaagagagaggagacattcaacaaacaagtatttattgagcgcctactatgtgccaggcactgttctagaccccccccagaagaaaaaacaaaaaacaagatagaggcagcaaacacaaattctgagggagaggaaaggggcagttgagtaagacggctaagggaactgagaagcctgaggtgatgggggctctgccttaggcctcctcttcggcctcctcaccgaaatcctcctcctcttctgcggtggcatcctggtactgctgatactcagagacgaggtcgttcatgttgctctcagcctcggtgaactccatctcgtccatgccctcgcctgtgtaccagtggaggaaggccttccggcggaacatggcagtgaactgctccgagatgcgcttgaagagctcctggatggctgtgctattgccaatgaaggtgactgccatcttgaggccacgaggtgggatgtcacagacggctgtcttgacattgttggggatccattccacaaagtagctgctgttcttgttctgcacgttaagcatctgctcatcgacctccttcatggacatccgaccacggaagacagcagccacggtgaggtatcggccgtggcgggggtcacaggcagccatcatgttcttggcatcgaagacctgctgggtgagttccggcactgtgagagctcgatactgctggcttccacggctggtgagaggggcaaagccaggcataaagaaatggagacgtgggaaggggaccatgttgactgccaacttgcggaggtcagcattgagctggccagggaaacggaggcaggtggtgac

SEQ ID NO: 155 - DNA, 36 bp, Homo sapiens
  misc_feature: P268H
atggtccccttcccacgtctccatttctttatgctt

SEQ ID NO: 156 - DNA, 36 bp, Homo sapiens; identical to SEQ ID NO: 130
  misc_feature: Mutated P268H

SEQ ID NO: 157 - DNA, 49 bp, Homo sapiens; identical to SEQ ID NO: 31
  misc_feature: F270V

SEQ ID NO: 158 - DNA, 49 bp, Homo sapiens; identical to SEQ ID NO: 32
  misc_feature: Mutated F270V

SEQ ID NO: 159 - DNA, 45 bp, Homo sapiens; identical to SEQ ID NO: 92
  misc_feature: A271P

SEQ ID NO: 160 - DNA, 45 bp, Homo sapiens; identical to SEQ ID NO: 93
  misc_feature: Mutated A271P

SEQ ID NO: 161 - DNA, 49 bp, Homo sapiens; identical to SEQ ID NO: 31
  misc_feature: T274I

SEQ ID NO: 162 - DNA, 49 bp, Homo sapiens; identical to SEQ ID NO: 36
  misc_feature: Mutated T274I

SEQ ID NO: 163 - DNA, 49 bp, Homo sapiens; identical to SEQ ID NO: 31
  misc_feature: T274P

SEQ ID NO: 164 - DNA, 49 bp, Homo sapiens
  misc_feature: Mutated T274P
atttctttatgcctggctttgcccctctccccagccgtggaagccagca

SEQ ID NO: 165 - DNA, 49 bp, Homo sapiens; identical to SEQ ID NO: 31
  misc_feature: R276H

SEQ ID NO: 166 - DNA, 49 bp, Homo sapiens; identical to SEQ ID NO: 40
  misc_feature: Mutated R276H

SEQ ID NO: 167 - DNA, 43 bp, Homo sapiens; identical to SEQ ID NO: 115
  misc_feature: V293D

SEQ ID NO: 168 - DNA, 43 bp, Homo sapiens; identical to SEQ ID NO: 116
  misc_feature: Mutated V293D

SEQ ID NO: 169 - DNA, 35 bp, Homo sapiens; identical to SEQ ID NO: 122
  misc_feature: A364T

SEQ ID NO: 170 - DNA, 35 bp, Homo sapiens; identical to SEQ ID NO: 123
  misc_feature: Mutated A364T